(19) United States
(12) Patent Application Publication
(10) Pub. No.: US 2013/0165751 A1
(43) Pub. Date: Jun. 27, 2013

(54) WATERPROOF ENDOSCOPE AND A METHOD OF MANUFACTURING THE SAME

(71) Applicants: Yi-Chen Chou, Tainan City (TW); Kai-Cheng Wang, Tainan City (TW)

(72) Inventors: Yi-Chen Chou, Tainan City (TW); Kai-Cheng Wang, Tainan City (TW)

(73) Assignees: HIMAX TECHNOLOGIES LIMITED, Tainan (TW); HIMAX IMAGING LIMITED, Tainan City (TW)

(21) Appl. No.: 13/335,769

(22) Filed: Dec. 22, 2011

(51) Int. Cl. A61B 1/04; B32B 38/04; B32B 37/12; C09J 5/00

(52) U.S. Cl. 600/109; 156/305; 156/250; 156/182

(57) ABSTRACT

The invention is directed to a waterproof endoscope having a wafer-level imaging module (WLM) bonded on a substrate. The endoscope also includes a holder, which has a groove formed into an inner surface of the holder. First glue is filled in the groove, and second glue is applied at a junction between the holder and the tube.

### BACKGROUND OF THE INVENTION

1. Field of the Invention

The present invention generally relates to an endoscope, and more particularly to a waterproof endoscope having a distal section made of a wafer-level module.

2. Description of Related Art

An endoscope is an instrument that is capable of being inserted into an organ to examine the interior of the organ. The endoscope generally includes a flexible tube; a light source disposed at a proximal end of the endoscope and the light is delivered via an optical fiber; and a lens system disposed at a distal end of the endoscope for collecting image that is transferred via an optical fiber.

Due to the miniature dimension of the endoscope, the manufacturing of the lens system requires great effort and thus making the overall cost high and accessibility low. Moreover, infection may probably occur due to inadequate cleaning of the endoscope. As far as the cost and infection are concerned, a single-use disposable endoscope may alleviate the problems mentioned above.

For the reason that the conventional endoscope is either high-priced or at the risk of infection, a need has arisen to propose a novel endoscope that eliminates the problems mentioned above.

### SUMMARY OF THE INVENTION

in view of the foregoing, the embodiment of the present invention provides an endoscope having a distal section made of a wafer-level imaging module with waterproof capability. The endoscope of the embodiment may be adaptable to disposable use to eliminate possibility of infection.

According to one embodiment of the invention, a waterproof endoscope includes a substrate, a wafer-level imaging module (WLM), a tube, a holder, first glue and second glue. The WLM is bonded on a first surface of the substrate, and the tube has a distal end coupled to a second surface of the substrate. The substrate with the bonded WLM is attached to the holder, wherein the holder has a groove formed into an inner surface of the holder, the groove being aligned with the WLM such that the groove faces a top surface of the WLM. The first glue is filled in the groove, and the second glue is applied at a junction between the holder and the tube.

According to another embodiment of the invention, a method of manufacturing a waterproof endoscope includes providing a substrate, a wafer-level imaging module (WLM), a holder and a tube. First glue is then filled in the groove formed into an inner surface of the holder. After attaching the WLM into the holder, second glue is applied at a junction between the holder and the tube.

### BRIEF DESCRIPTION OF THE DRAWINGS

FIG. 1A, FIG. 1B and FIG. 1C show a top view, a lateral cross-sectional view and a bottom view, respectively, of a distal section of a waterproof endoscope according to one embodiment of the present invention;

FIG. 2 shows a perspective view of the distal section of the waterproof endoscope according to the embodiment of the invention;

FIG. 3 schematically shows a perspective view of the WLM of FIG. 1B; and

FIG. 4 shows a flow diagram illustrating a method of manufacturing a waterproof endoscope according to one embodiment of the present invention.

### DETAILED DESCRIPTION OF THE INVENTION

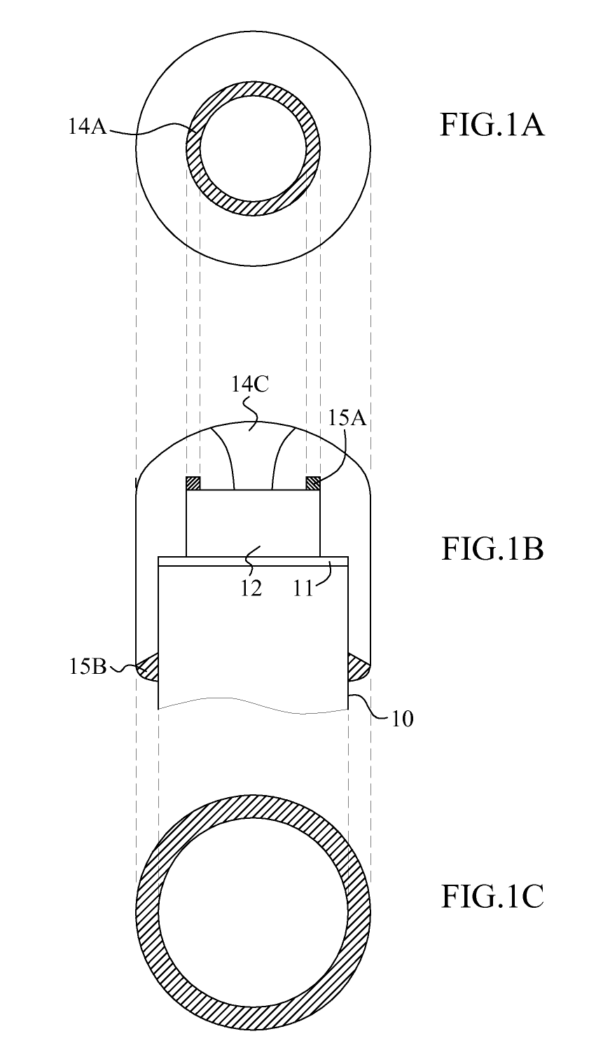

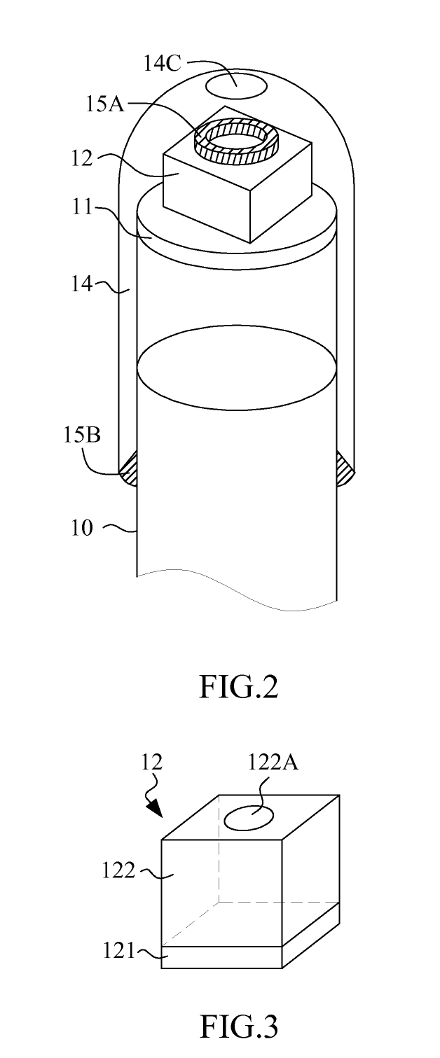

FIG. 1A, FIG. 1B and FIG. 1C show a top view, a lateral cross-sectional view and a bottom view, respectively, of a distal section of a waterproof endoscope according to one embodiment of the present invention. FIG. 2 shows a perspective view of the distal section of the waterproof endoscope according to the embodiment of the invention. The distal section of the endoscope is disposed at and coupled to a distal end of a tube 10 of the endoscope.

In the embodiment, the distal section primarily includes a substrate 11, a wafer-level imaging module (or wafer-level module, WLM, for short) 12 and a holder 14. Specifically, the WLM 12 is bonded on a first (top) surface of the substrate 11 (such as a printed circuit board, PCB), for example, by reflow soldering, and the substrate 11 with the bonded WLM 12 is then attached to the holder 14. Subsequently, the distal end of the tube 10 is coupled to a second (bottom) surface of the substrate 11. In this specification, “top” is directed to the distal end of the endoscope, and “bottom” is directed to the proximal end of the endoscope. The holder 14 of the embodiment is usually made of opaque material. In addition to the space provided for accommodating the WLM 12 and the distal end of the tube 10, the holder 14 may further have an opening 14C situated above and aligned with the WLM 12.

FIG. 3 schematically shows a perspective view of the WLM 12. In the embodiment, the WLM 12 includes a wafer-level image sensor 121 and wafer-level optics (WLO) 122. The wafer-level image sensor 121 is situated facing the substrate 11, and may be, but not limited to, a complementary metal oxide semiconductor (CMOS) image sensor (commonly abbreviated as CIS). The wafer-level optics 122, such as a lens, is situated away from the substrate 11, and may be made of, but not limited to, glass. The wafer-level optics 122 has an opening 122A, which acts as the aperture of a diaphragm, through which light controllably passes and finally rests on the wafer-level image sensor 121. The structure of the wafer-level optics 122 may be referred, for example, to US Patent Publication No. 2011/0249176 to Hsueh, entitled “WAFER LEVEL LENS MODULE AND RELATED METHOD FOR FORMING THE SAME,” the disclosure of which is incorporated herein by reference. The wafer-level image sensor 121 and the wafer-level optics 122 may be bonded together, for example, with an adhesive. Compared to the conventional endoscope, the endoscope of the present embodiment makes use of the mass-productivity and low cost of semiconductor technique to manufacture the imaging system of the endoscope, and is adaptable to the disposable endoscope to eliminate possibility of infection. Wafer-level (or wafer-scale) module is a technique of fabricating miniaturized optics such as lens module or camera module at the wafer level using semiconductor techniques, and details of manufacturing the wafer-level imaging module 12 may be referred, for example, to U.S. Pat. No. 7,564,496 to Wolterink et al., entitled “Camera device, method of manufacturing a camera device, wafer scale package,” the disclosure of which is incorporated herein by reference.

According to one aspect of the embodiment, the holder 14 has a groove (or cannelure) 14A cut into an inner surface of the holder 14. The groove 14A may be, but not limited to, a circular ring. The groove 14A is aligned with the WLM 12 such that the groove 14A faces the top surface of the WLM 12. After the WLM 12 is attached to the holder 14, the opening 122A of the wafer-level optics 122 is surrounded within the groove 14A and is substantially aligned, with the opening 14C of the holder 14.

Before the WLM 12 is attached to the holder 14, the groove 14A is filled with (first) glue 15A. Therefore, the assembled endoscope (as shown in FIG. 1B) is waterproofed such that liquid

the organ to be examined) cannot be leaked into the distal section of the endoscope via the opening 14C. It is noted that the groove 14A should be preferably distanced from the opening 14C with a sufficient distance such that the filled glue 15A may not be leaked, for example, into the organ while examining the interior of the organ.

According to another aspect of the embodiment, (second) glue 15B is further applied at the junction between the holder 14 and the tube 10, rendering the endoscope being waterproofed. As shown in FIG. 1B, the edge of the holder 14 at the junction between the holder 14 and the tube 10 is indented with an inclined angle, thereby forming a space to accommodate the glue 15B.

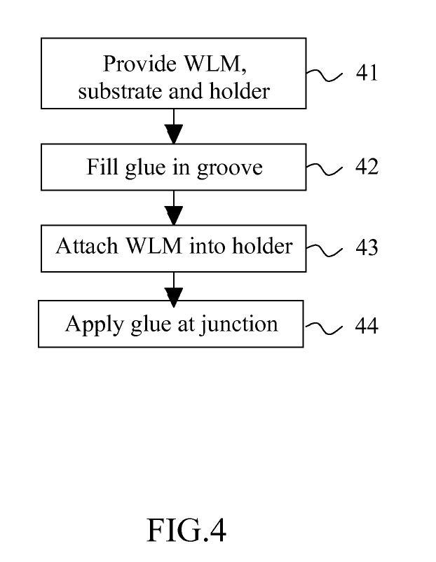

FIG. 4 shows a flow diagram illustrating a method of manufacturing a waterproof endoscope according to one embodiment of the present invention. In step 41, the substrate 11, the WLM 12 and the holder 14 are provided, wherein the WLM 12 is bonded on the substrate 11, for example, by reflow soldering. Subsequently, in step 42, the glue 15A is filled in the groove 14A, followed by attaching the WLM 12 into the holder 14 (step 43). Accordingly, the glue 15A makes the endoscope waterproof by keeping liquid from being leaked into the distal section of the endoscope via the opening 14C. Finally, in step 44, the glue 15B is further applied at the junction between the holder 14 and the tube 10, thereby rendering the endoscope being waterproofed.

Although specific embodiments have been illustrated and described, it will be appreciated by those skilled in the art that various modifications may be made without departing from the scope of the present invention, which is intended to be limited solely by the appended claims.

What is claimed is:

1. A waterproof endoscope, comprising:
a substrate;
a wafer-level imaging module (WLM) bonded on a first surface of the substrate;
a tube having a distal end coupled to a second surface of the substrate;
a holder, to which the substrate with the bonded WLM is attached, wherein the holder has a groove formed into an inner surface of the holder, the groove being aligned with the WLM such that the groove faces a top surface of the WLM;
first glue filled in the groove; and
second glue applied at a junction between the holder and the tube.

2. The waterproof endoscope of claim 1, wherein the WLM comprises:
a wafer-level image sensor; and
a wafer-level optics bonded with the wafer-level image sensor;
wherein the wafer-level image sensor is situated facing the substrate, and the wafer-level optics is situated away from the substrate.

3. The waterproof endoscope of claim 2, wherein the wafer-level image sensor comprises a complementary metal oxide semiconductor (CMOS) image sensor.

4. The waterproof endoscope of claim 2, wherein the wafer-level optics comprises a lens.

5. The waterproof endoscope of claim 4, wherein the lens is made of glass.

6. The waterproof endoscope of claim 1, wherein the substrate comprises a printed circuit board.

7. The waterproof endoscope of claim 1, wherein an edge of the holder at the junction between the holder and the tube is indented with an inclined angle, thereby forming a space to accommodate the second glue.

8. The waterproof endoscope of claim 1, wherein the holder is made of opaque material.

9. The waterproof endoscope of claim 1, wherein the holder has an opening situated above the WLM.

10. A method of manufacturing a waterproof endoscope, comprising:
providing a substrate, a wafer-level imaging module (WLM), a holder and a tube, wherein the WLM is bonded on a first surface of the substrate, the tube is coupled to a second surface of the substrate, and the holder has a groove formed into an inner surface of the holder;
filling first glue in the groove;
attaching the WLM into the holder; and
applying second glue at a junction between the holder and the tube.

11. The method of claim 10, further comprising a step of aligning the groove with the WLM such that the groove faces a top surface of the WLM.

12. The method of claim 10, wherein the WLM comprises:
a wafer-level image sensor; and
a wafer-level optics bonded with the wafer-level image sensor;
wherein the wafer-level image sensor is situated facing the substrate, and the wafer-level optics is situated away from the substrate.

13. The method of claim 12, wherein the wafer-level image sensor comprises a complementary metal oxide semiconductor (CMOS) image sensor.

14. The method of claim 12, wherein the wafer-level optics comprises a lens.

15. The method of claim 10, wherein the substrate comprises a printed circuit board.

16. The method of claim 10, further comprising a step of indenting an edge of the holder at the junction between the holder and the tube to form an inclined angle, thereby forming a space to accommodate the second glue.

17. The method of claim 10, wherein the holder is made of opaque material.

18. The method of claim 10, further comprising a step of forming an opening in the holder, the opening being situated above the WLM.
